(19) United States
(12) Patent Application Publication
(10) Pub. No.: US 2012/0071499 A1
(43) Pub. Date: Mar. 22, 2012

(54) Substituted Spiro[3H-Indole-3,6'(5'H)-[1H]Pyrrolo[1,2c]Imidazole-1',2(1H,2'H)-diones

(76) Inventors: Xin-Jie Chu, Livingston, NJ (US); Qingjie Ding, Bridgewater, NJ (US); Nan Jiang, Pine Brook, NJ (US); Jin-Jun Liu, Warren Township, NJ (US); Jing Zhang, Parsippany, NJ (US); Zhuming Zhang, Hillsborough, NJ (US)

(21) Appl. No.: 13/212,294

(22) Filed: Aug. 18, 2011

Related U.S. Application Data
(60) Provisional application No. 61/384,416, filed on Sep. 20, 2010.

(51) Int. Cl. A61K 31/4439; C07D 487/20; A61K 31/4188

(52) U.S. Cl. 514/278; 514/393; 546/15; 548/301.1

(57) ABSTRACT

There are provided compounds of the formula

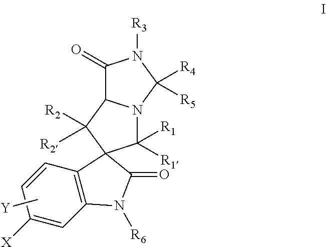

wherein X, Y, R₁, R_{1′}, R₂, R_{2′}, R₃, R₄, R₅ and R₆ are as described herein and enantiomers or a pharmaceutically acceptable salt or ester thereof. The compounds are useful as anticancer agents.

### PRIORITY TO RELATED APPLICATION(S)

This application claims the benefit of U.S. Provisional Application No. 61/384,416, filed Sep. 20, 2010, which is hereby incorporated by reference in its entirety.

### FIELD OF THE INVENTION

The present invention relates to substituted spiro[3H-indole-3,6′(5′H)-[1H]pyrrolo[1,2-c]imidazole-1′,2(1H,2′H)-dione derivatives which act as inhibitors of MDM2-p53 interactions and are useful in the amelioration or treatment of cancer.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells.

The present invention relates to spiro pyrrolo dione derivatives I which act as antagonists of mdm2 interactions and hence are useful as potent and selective anticancer agents.

### SUMMARY OF THE INVENTION

The present compounds are of the general formula

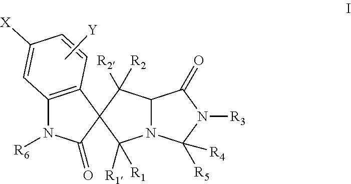

wherein X, Y, R₁, R_{1′}, R₂, R_{2′}, R₃, R₄, R₅ and R₆ are as described herein
and enantiomers or a pharmaceutically acceptable salt or ester thereof.

### DETAILED DESCRIPTION OF THE INVENTION

There are provided compounds of the formula

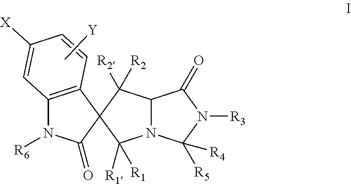

wherein
X is selected from the group consisting of H, F, Cl, Br, I, cyano, nitro, lower alkyl, lower alkynyl and lower alkoxy.

### Y is H or F;

R₁ and R_{1′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ and R_{2′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and enantiomers thereof or a pharmaceutically acceptable salt thereof.

More preferred are compounds of formula I in which R_{2′} is H, R₂ is selected from the group consisting of a substituted phenyl as shown in formula II:

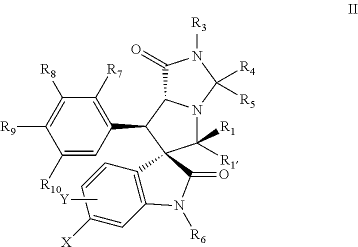

wherein,
X is selected from the group consisting of H, F, Cl and Br, I, cyano, nitro, lower alkyl, lower alkynyl and lower alkoxy;

### Y is H or F;

R₈ is selected from the group consisting of F, Cl and Br;
R₇, R₉ and R₁₀ are H or F with the proviso that at least two of R₇, R₉ and R₁₀ are hydrogen;
R₁ and R_{1′} are independently selected from the group consisting of hydrogen, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle, and substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and the enantiomers thereof or a pharmaceutically acceptable salt thereof.

Further preferred are compounds of formula II in which R_{1′} is hydrogen, R₁ is selected from a group consisted of substituted lower alkyl shown as in formula III:

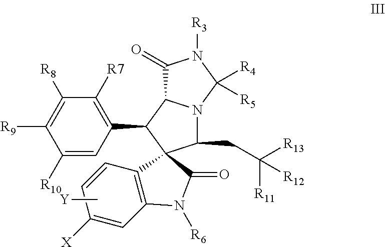

wherein,
X is selected from the group consisting of H, F, Cl and Br, I, cyano, nitro, lower alkyl, lower alkynyl and lower alkoxy;

### Y is H or F;

R₈ is selected from the group consisting of F, Cl and Br;
R₇, R₉, R₁₀ are selected from H or F with the proviso that at least two of R₇, R₉ and R₁₀ are hydrogen;
R₁₁, R₁₂ are both methyl, or linked to form a cyclopropyl, cyclobutyl, cyclopentyl or cyclohexyl group;
R₁₃ is (CH₂)ₘ—R₁₄;
m is selected from 0, 1 or 2;
R₁₄ is selected from hydrogen, hydroxyl, lower alkyl, lower alkoxy, lower cycloalkenyl, substituted cycloalkenyl, lower cycloalkyl, substituted cycloalkyl, aryl, substituted aryl, hetereoaryl, substituted heteroaryl, hetereocycle or substituted heterocycle;
R₃ is aryl, substituted aryl, heteroaryl or substituted heteroaryl;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and enantiomers thereof or a pharmaceutically acceptable salt or ester thereof.

Further preferred are compounds of formula III in which R₁₁, R₁₂, R₁₃ are methyl as shown in formula IV,

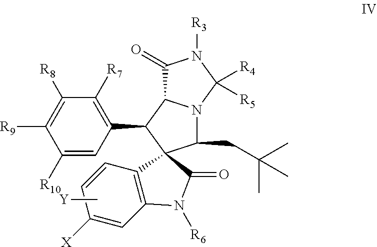

wherein,
X is selected from the group consisting of H, F, Cl and Br, I, cyano, nitro, lower alkyl, lower alkynyl and lower alkoxy;

### Y is H or F;

R₈ is selected from the group consisting of F, Cl and Br;
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and the enantiomers thereof or a pharmaceutically acceptable salt thereof.

Further preferred are compounds of formula IV in which R₆ is H as shown in formula V.

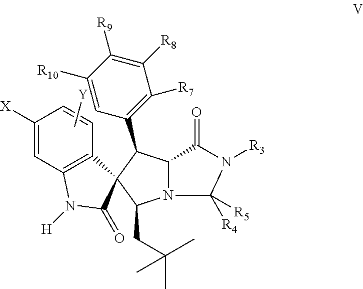

wherein,
X is selected from the group consisting of F, Cl and Br
Y is a mono substituting group consisting of H or F;
R₈ is selected from the group consisting of F, Cl and Br,
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, cyano, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
and the enantiomers thereof and a pharmaceutically acceptable salt thereof.

Compounds prepared according to the invention include:
rac 5-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)picolinamide
rac-4-((3R,5′S,7′S,7a′R)-6-chloro-7′-(3-chloro-2-fluorophenyl)-5′-(2,2-dimethyl-propyl)-1′,2-dioxo-3′-thioxo-7′,7a′-dihydro spiro[indoline-3,6′-pyrrolo[1,2-c]imidazole]-2′(1′H,3′H,5′H)-yl)benzonitrile,
rac-4-((3R,5′S,7′S,7a′R)-6-chloro-7′-(3-chloro-2-fluorophenyl)-5′-(2,2-dimethyl-propyl)-1′,2,3′-trioxo-7′,7a′-dihydrospiro[indoline-3,6′-pyrrolo[1,2-c]imidazole]-2′(1′H,3′H,5′H)-yl)benzonitrile,
chiral (3R,5′S,7′S,7a′R)-6-chloro-7′-(3-chloro-2-fluorophenyl)-2′-[4-(2-hydroxyethoxy)-2-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione,
chiral (3R,5′S,7′S,7a′R)-6-chloro-7′-(3-chloro-2-fluorophenyl)-2′-[4-(2-hydroxyethoxy)-3-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione,
rac4-((3R,5′S,7′S,7a′R)-6-chloro-7′-(3-chloro-2-fluorophenyl)-1-(hydroxymethyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
Rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)2-methoxybenzoic acid,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)2-methoxybenzoic acid,
chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid,
chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzamide,
chiral 4-((3R,5′S,7′S,7a′R)-6-chloro-7′-(3-chloro-4-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid,
racl 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzamide,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
4-((3R,5′S,7′S,7a′R)-6-chloro-7′-(3-chloro-2-fluorophenyl)-1-(hydroxymethyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
methyl rac-4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoate,
methyl rac-4-((3R,5′S,7′S,7a′R)-6-chloro-7′-(2,5-difluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-fluorobenzoate,
rac 2-chloro-4-((3R,5′S,7′R,7a′R)-7′-(3-chlorophenyl)-6-fluoro-5′-neopentyl-1′,2-dioxo-7′,7a′-dihydrospiro[indoline-3,6′-pyrrolo[1,2-c]imidazole]-2′(1′H,3′H,5′H)-yl)benzamide,
rac 2-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)acetic acid,
rac 5-{[(2′S,3′R,4′S,5′R)-6-Chloro-4′-(3-chloro-2-fluoro-phenyl)-2′-(2,2-dimethylpropyl)-2-oxo-1,2-dihydro-spiro[indole-3,3′-pyrrolidin]e-5′-carbonyl]-amino}-pyridine-2-carboxylic acid methyl ester,
rac 5-{[(2′S,3′R,4′S,5′R)-6-Chloro-4′-(3-chloro-2-fluoro-phenyl)-2′-(2,2-dimethylpropyl)-2-oxo-1,2-dihydro-spiro[indole-3,3′-pyrrolidin]e-5′-carbonyl]-amino}-pyridine-2-carboxylic acid,
4-((3S,3′S,5′S,7′R,7a′R)-6-chloro-7′-(3-chloro-4-fluorophenyl)-3′-cyclopropyl-5′-neopentyl-1′,2-dioxo-7′,7a′-dihydrospiro[indoline-3,6′-pyrrolo[1,2-c]imidazole]-2′(1′H,3′H,5′H)-yl)benzamide,
rac 4-((3S,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide,
4-((3R,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′ (1′H,3′H,5′H)-yl)benzamide and
rac 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid hydrochloride.

### TERMS & DEFINITIONS

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. In the specification where indicated the various groups may be substituted by preferably, 1-3 substituents independently selected from the group consisting of H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle.

The term “alkyl” refers to straight- or branched-chain saturated hydrocarbon groups having from 1 to about 20 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, and in certain embodiments from 1 to 4 carbon atoms. Examples of alkyl groups include, but are not limited to, methyl, ethyl, n-propyl, i-propyl, n-butyl, s-butyl, t-butyl, n-pentyl, and s-pentyl.

As used herein, “cycloalkyl” is intended to refer to any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “alkenyl” as used herein means an unsaturated straight-chain or branched aliphatic hydrocarbon group containing one double bond and having 2 to 6, preferably 2 to 4 carbon atoms. Examples of such “alkenyl group” are vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “halogen” as used in the definitions means fluorine, chlorine, bromine, or iodine, preferably fluorine and chlorine.

“Aryl” means a monovalent, monocyclic or bicyclic, aromatic carbocyclic hydrocarbon radical, preferably a 6-10 member aromatic ring system. Preferred aryl groups include, but are not limited to, phenyl, naphthyl, tolyl, and xylyl.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole and tetrazolyl.

In the case of aryl or heteroaryl which are bicyclic it should be understood that one ring may be aryl while the other is heteroaryl and both being substituted or unsubstituted.

“Heterocycle” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like.

“Hetero atom” means an atom selected from N, O and S.

“Alkoxy, alkoxyl or lower alkoxy” refers to any of the above lower alkyl groups attached to an oxygen atom. Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (6th Ed. 1995) at pp. 196 and 1456-1457.

The compounds of formulas I-V as well as their salts that have at least one asymmetric carbon atom may be present as racemic mixtures or different stereoisomers. The various isomers can be isolated by known separation methods, e.g., chromatography.

Compounds disclosed herein and covered by formulas I-V above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

The compounds of the present invention are useful in the treatment or control of cell proliferative disorders, in particular oncological disorders. These compounds and formulations containing said compounds may be particularly useful in the treatment or control of solid tumors, such as, for example, breast, colon, lung and prostate tumors.

A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

“Effective amount” means an amount that is effective to prevent, alleviate or ameliorate symptoms of disease or prolong the survival of the subject being treated.

“IC₅₀” refers to the concentration of a particular compound required to inhibit 50% of a specific measured activity. IC₅₀ can be measured, inter alia, as is described subsequently.

### General synthesis of substituted spiro[3H-indole-3,6′(5′H)-[1H]pyrrolo[1,2-c]imidazole-1′,2(1H,2′H)-diones

The present invention provides methods for the synthesis of the substituted spiro[3H-indole-3,6′(5′H)-[1H]pyrrolo[1,2-c]imidazole-1′,2(1H,2′H)-diones of the invention.

The compounds of the invention can be prepared by processes known in the art. Suitable processes for synthesizing these compounds are also provided in the examples. Generally, compounds of formula I can be synthesized according to one of the below described synthetic routes.

The key transformations are a convergent [2+3] cycloaddition of imine A and activated olefin B to generate compounds C in a stereoselective manner. Compound C then can be used directly to make amide D or resolved first and then used to make chiral amide D. Compound D was then reacted with aldehyde to generate the desired tagrget I.

The starting materials are either commercially available or can be synthesized by methods known to those of ordinary skill in the art. Preparations of intermediates A and B are illustrated in Schemes 1 and 2 below. In general an appropriately selected aldehyde or ketone can be reacted with glycine tert-butyl ester or glycine methyl ester to generate imine A as a crude product (see Scheme 1 below).

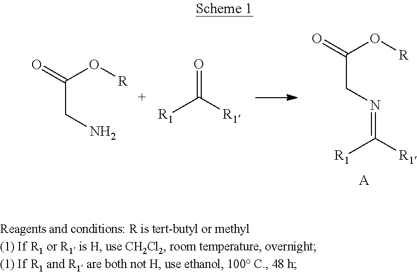

An intermediate of formula B can be made from a base-catalyzed condensation reaction of appropriately selected substituted-2-indolone and aldehydes. The reaction proceeds in good yield as described in scheme 2 below.

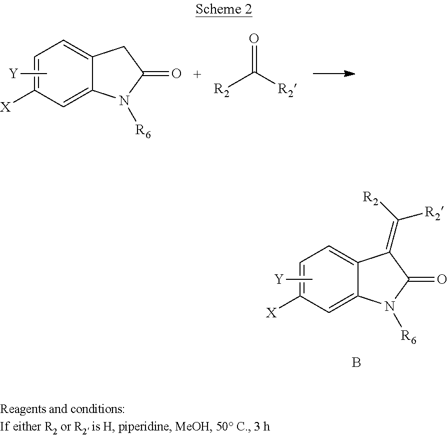

As is illustrated in Scheme 3 below, spiro-indole pyrrolidines of formula C can be made from intermediates A and B by a convergent 1,3-dipolar cycloaddition reaction mediated by lewis acid AgF and triethylamine, immediately followed by an isomerization reaction using DBU as the base in tert-butanol at an elevated temperature of 100° C. to 150° C. then followed by hydrolysis. The [2+3] cycloaddition reactions of azomethine ylides 1,3-dipoles (that were generated from reacting intermediate A with AgF) with olefinic dipolarphiles to form pyrrolidine ring are described in the literature, including Jorgensen, K. A. et al (Org. Lett. 2005, Vol 7, No. 21, 4569-4572), Grigg, R. et al (Tetrahedron, 1992, Vol 48, No. 47, 10431-10442; Tetrahedron, 2002, Vol 58, 1719-1737), Schreiber, S. L. et al (J. Am. Chem. Soc., 2003, 125, 10174-10175), and Carretero, J. C. et al (Tetrahedron, 2007, 63, 6587-6602). Compounds of formula C are subsequently converted to compounds of formula D by amide formation with various amines using HATU as the coupling reagent. Other known arts using different activating agents like EDCI, HOBT or oxylyl chloride also work.

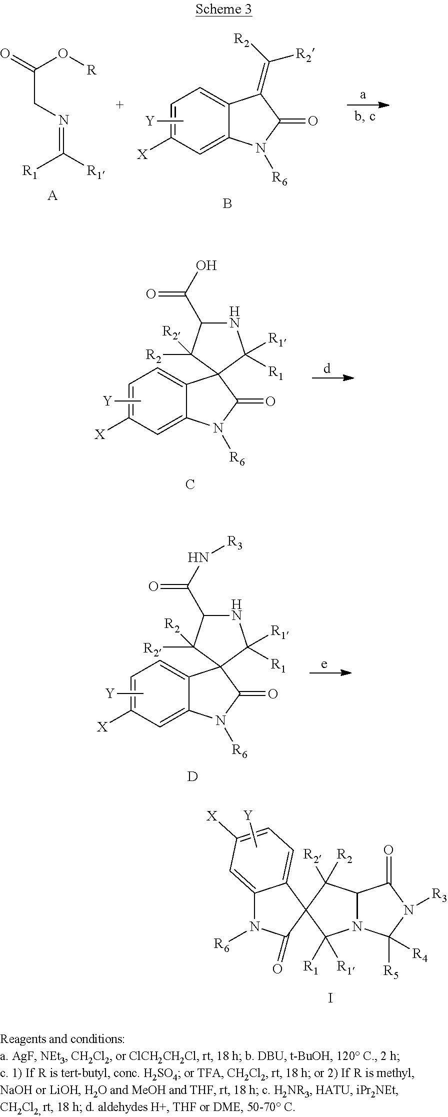

Racemic C can be readily resolved into two optically pure or enriched chiral enantiomers C1 and C2 by separation using chiral Super Fluid Chromatography (SFC). (see Scheme 4 below) and racemic D can be resolved by a similar manner (see Scheme 5 below). Even formula I can also be resolved by a similar method.

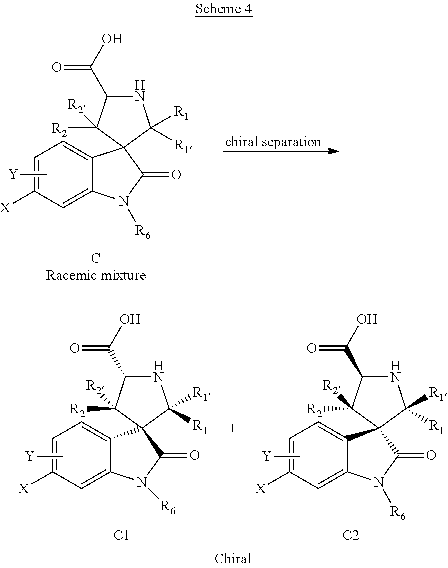

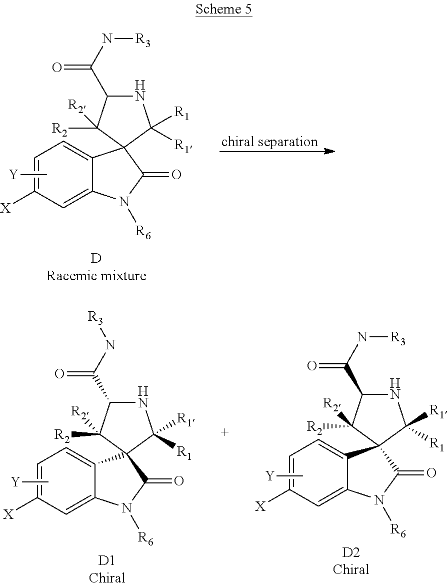

Resolution methods are well known, and are summarized in “Enantiomers, Racemates, and Resolutions” (Jacques, J. et. al. John Wiley and Sons, NY, 1981). Methods for chiral HPLC are also well known, and are summarized in “Separation of Enantiomers by Liquid Chromatographic Methods” (Rirkle, W. H. and Finn, J in Asymmetric Synthesis” Vol. 1, Morrison, J. D., Ed. Academic Press, In., NY 1983, pp. 87-124).

An alternative method to make formula D can be achieved by an addition reaction of imine E and alkene B as described in Scheme 6.

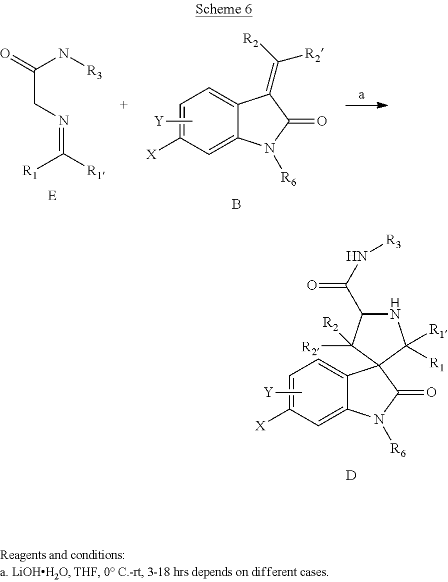

In the case where R₄ and R₅ together form an oxo or thioxo group, compounds of formula I can generally be prepared by the procedure described in Scheme 7.

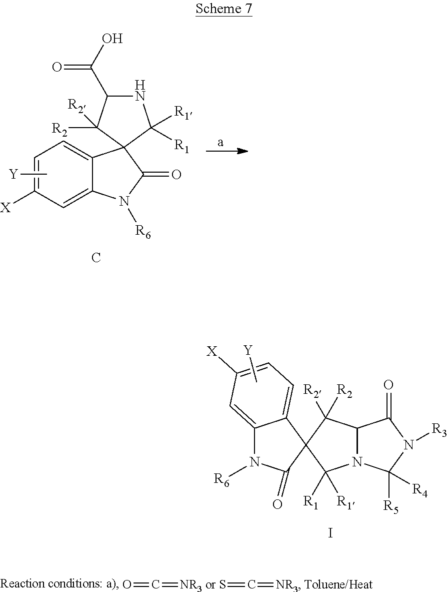

Converting a Compound of Formulas I-V that Bears a Basic Nitrogen into a Pharmaceutically Acceptable Acid Addition Salt

The optional conversion of a compound of formula I that bears a basic nitrogen into a pharmaceutically acceptable acid addition salt can be effected by conventional means. For example, the compound can be treated with an inorganic acid such as for example hydrochloric acid, hydrobromic acid, sulfuric acid, nitric acid, phosphoric acid, or with an appropriate organic acid such as acetic acid, citric acid, tartaric acid, methanesulfonic acid, p-toluene sulfonic acid, or the like.

Converting a Compound of Formulas I-V that Bears a Carboxylic Acid Group into a Pharmaceutically Acceptable Alkali Metal Salt

The optional conversion of a compound of formula I that bears a carboxylic acid group into a pharmaceutically acceptable metal salt can be effected by conventional means. For example, the compound can be treated with an inorganic base such as lithium hydroxide, sodium hydroxide, potassium hydroxide, or the like.

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using Isis AutoNom 2000.

### ABBREVIATIONS USED IN THE EXAMPLES

### HRMS: High Resolution Mass Spectrometry

### LCMS: Liquid Chromatography Mass Spectrometry

HATU: 2-(7-Azabenzotriazol-1-yl)-n,n,n′,n′-tetramethyluronium hexafluorophosphate
RT (or rt) Room temperature
DBU: 1,8-Diazabicyclo[5,4,0]undec-7-ene
DIBAL: Diisobutylalumiunum hydride
iPA: Isopropyl alcohol
ASDI: ASDI-Intermediates (company name)
RP-HPLC: Reverse phase HPLC

### Min: Minutes

### H or hrs: Hours

### GST; Glutathione S-transferase

TRF: Time resolved fluorescensce

### Example 1

### Preparation of intermediate [3,3-dimethyl-but-(E)-ylideneamino]-acetic acid tert-butyl ester

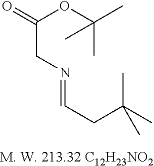

A mixture of glycine tert-butyl ester (Alfa) (2.71 g, 20.0 mmol) and 3,3-dimethyl-butyraldehyde (Alfa) (2.21 g, 21.0 mmol) in CH₂Cl₂ (50 mL) was stirred at rt overnight. The reaction mixture was concentrated and the residue was dried in vacuo to give [3,3-dimethyl-but-(E)-ylideneamino]-acetic acid tert-butyl ester (4.29 g, 100%) as colorless oil which was used in the next step without further purification.

### Example 2

### Preparation of intermediate E/Z-6-chloro-3-(3-chloro-2-fluoro-benzylidene)-1,3-dihydro-indol-2-one

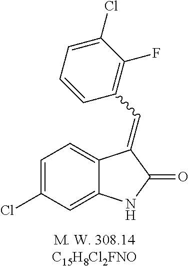

To the mixture of 6-chloro-2-oxindole (11 g, 65.6 mmol) (Crescent) and 3-chloro-2-fluorobenzaldehyde (12 g, 75.7 mmol) (Aldrich) in methanol (140 mL) was added piperidine (5.59 g, 65.6 mmol) (Aldrich) dropwise. The mixture was then heated at 50° C. for 3 h. After cooled to 4° C., the mixture was filtered and resulting precipitate was collected, dried to give E/Z-6-chloro-3-(3-chloro-2-fluoro-benzylidene)-1,3-dihydro-indol-2-one as a yellow solid (Yield 18 g, 89%).

### Example 3

### Preparation of intermediate rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester

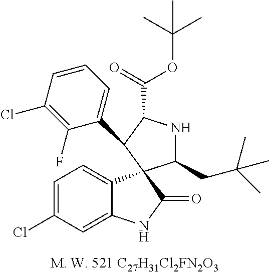

To a solution of [3,3-dimethyl-but-(E)-ylideneamino]-acetic acid tert-butyl ester (3.37 g, 15.8 mmol) prepared in Example 1 and E/Z-6-chloro-3-(3-chloro-2-fluoro-benzylidene)-1,3-dihydro-indol-2-one (4 g, 13 mmol) prepared in Example 2 in dichloromethane (100 mL) were added triethyl amine (6.6 mL, 47.4 mmol) and AgF (2 g, 15.8 mmol). The mixture was stirred at room temperature for 18 h. The mixture was concentrated, and the residue was partitioned between ethyl acetate and brine. The organic layer was separated, washed with water, dried over MgSO₄, and concentrated. The residue was dissolved into t-butanol (30 mL), and DBU (7.2 g, 47.4 mmol) was added. The reaction mixture was heated at 120° C. for 2 h. The mixture was then cooled to room temperature and concentrated. The residue was partitioned between ethyl acetate and water. The organic layer was separated, and the aqueous layer was extracted with ethyl acetate. The organic layers were combined, washed with water, brine, dried over MgSO₄, and concentrated. The residue was purified by chromatography (EtOAc:hexanes=1:3, 1:2) to give as rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester a white foam (2.7 g, 33%)

### Example 4

### Preparation of intermediate rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid

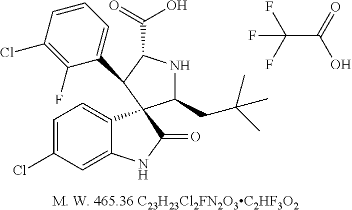

A solution of rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester (2.6 g, 4.8 mmol) in dichloromethane (60 mL) was added trifluoroacetic acid (8 mL). The reaction mixture was stirred at room temperature for 18 h, then concentrated. The residue was then triturated with ethyl ether hexanes, concentrated, dried in vacuo to give rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid as a off white solid (2.8 g, 93%).

### Example 5

### Preparation of rac-4-((3R,5′S,7′S,7a′R)-6-chloro-7′-(3-chloro-2-fluorophenyl)-5′-(2,2-dimethyl-propyl)-1′,2-dioxo-3′-thioxo-7′,7a′-dihydrospiro[indoline-3,6′-pyrrolo[1,2-c]imidazole]-2′(1′H,3′H,5′H)-yl)benzonitrile

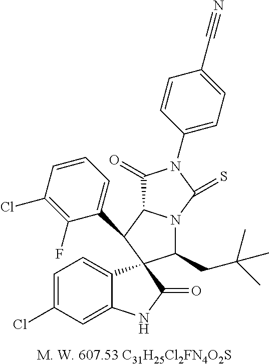

To a suspension of rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid (0.6 g, 1.04 mmol) in toluene (15 mL) was added triethylamine (0.31 g, 3.11 mmol), 4-isothiocyanatobenzonitrile (Aldrich) (0.5 g, 3.11 mmol) respectively. The mixture was heated at 110° C. for 0.5 h. The mixture was cooled to room temperature and concentrated. The residue was purified by chromatography (10-30% of EtOAc in hexanes) to give rac-4-((3R,5′S,7′S,7a′R)-6-chloro-7′-(3-chloro-2-fluorophenyl)-5′-(2,2-dimethyl-propyl)-1′,2-dioxo-3-thioxo-7′,7a′-dihydrospiro[indoline-3,6′-pyrrolo[1,2-c]imidazole]-2′(1′H,3′H,5′H)-yl)benzonitrile as a white solid (Yield 0.46 g, 73%).

LCMS (ES⁺) m/z Calcd for C₃₁H₂₅Cl₂FN₄O₂S+H [(m+H)⁺]:607, found: 607.

### Example 6

### Preparation of rac-4-((3R,5′S,7′S,7a′R)-6-chloro-7′-(3-chloro-2-fluorophenyl)-5′-(2,2-dimethyl-propyl)-1′,2,3′-trioxo-7′,7a′-dihydrospiro[indoline-3,6′-pyrrolo[1,2-c]imidazole]-2′(1′H,3′H,5′H)-yl)benzonitrile

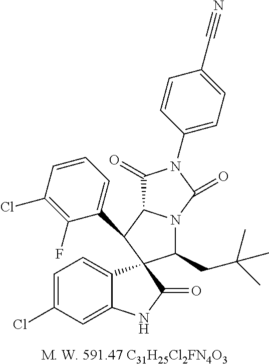

To a suspension of rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid prepared in Example 4 (0.25 g, 0.43 mmol) in toluene (15 mL) was added triethylamine (0.13 g, 1.29 mmol), 4-isocyanatobenzonitrile (Aldrich) (0.19 g, 1.29 mmol) respectively. The mixture was heated at 110° C. for 0.5 h. The mixture was cooled to room temperature and concentrated. The residue was purified by chromatography (20-40% of EtOAc in hexanes) to give rac-4-((3R,5′S,7′S,7a′R)-6-chloro-7′-(3-chloro-2-fluorophenyl)-5′-(2,2-dimethyl-propyl)-1′,2,3′-trioxo-7′,7a′-dihydrospiro[indoline-3,6′-pyrrolo[1,2-c]imidazole]-2′(1′1-1,3′H,5′H)-yl)benzonitrile as a white solid (Yield 0.14 g, 55%).

LCMS (ES⁺) m/z Calcd for C₃₁H₂₅Cl₂FN₄O₃+H[(M+H)⁺]: 591, found: 591.

### Example 7

### Preparation of intermediate chiral-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester

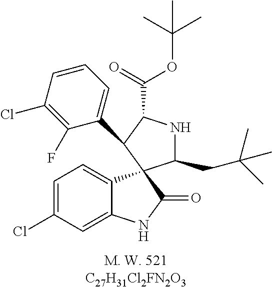

Rac-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester prepared in Example 3 (8.2 g) was separated by chiral SFC chromatography to provide chiral (2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester as a white solid (3.5 g, 43%) and chiral-(2′R,3′S,4′R,5′S)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester as a white solid (3.7 g, 45%).

### Example 8

### Preparation of intermediate chiral-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid

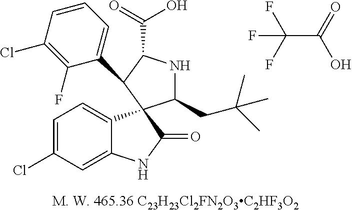

A solution of chiral-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid tert-butyl ester (3.5 g, 4.8 mmol) in dichloromethane (10 mL) was added trifluoroacetic acid (20 mL). The reaction mixture was stirred at room temperature for 18 h, then concentrated. The residue was then triturated with ethyl ether hexanes, concentrated, dried in vacuo to give chiral-(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid as a off white solid (3.8 g, 98%).

### Example 9

### Preparation of intermediate 4-[2-(tert-butyl-dimethyl-silanyloxy)-ethoxy]-2-methoxy-phenylamine

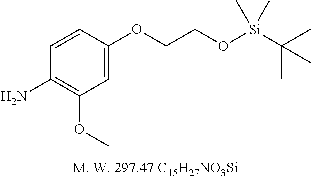

### Step A.

To a solution of 4-fluoro-2-methoxy-1-nitrobenzene (Combi-blocks) (3.4 g, 19.9 mmol) in DMSO (40 mL) was added an aqueous solution (1N) of NaOH (40 mL, 40 mmol). The reaction mixture was heated at 80° C. for 20 h. The mixture was cooled to room temperature, and the “pH” of the solution was adjusted to 5 by aqueous HCl solution. The mixture was extracted with ethyl acetate three times. The combined organic extract was washed with water, brine, dried over MgSO₄, and concentrated to give 3-methoxy-4-nitrophenol as a light yellow solid (3.2 g, 95%).

### Step B.

To a solution of 3-methoxy-4-nitrophenol (1 g, 5.9 mmol) in anhydrous DMF (25 mL) were added K₂CO₃ (2.45 g, 17.7 mmol) and (2-bromoethoxy)(tert-butyl)dimethylsilane (1.7 g, 7.1 mmol) sequentially. The reaction mixture was heated at 70° C. for 20 h. The mixture was cooled to room temperature, and diluted with water. The mixture was extracted with ethyl acetate three times. The combined organic extract was washed with water, brine, dried over MgSO₄, and concentrated. The residue was purified by chromatography (0-20% EtOAc in hexanes) to give tert-butyl-[2-(3-methoxy-4-nitro-phenoxy)-ethoxy]-dimethyl-silane as a light yellow oil (1.0 g, 52%).

### Step C.

A suspension of tert-butyl-[2-(3-methoxy-4-nitro-phenoxy)-ethoxy]-dimethyl-silane (1 g, 3.05 mmol) and Pd/C (Aldrich, 10%, 0.1 g) in ethyl acetate (25 mL) was vigorously shaken in a Parr under atmosphere of H₂ (50 psi) for 0.5 h. The mixture was filtered through a short pad of celite. The filtrate was concentrated to give 4-[2-(tert-butyl-dimethyl-silanyloxy)-ethoxy]-2-methoxy-phenylamine as a light yellow oil (0.9 g, 99%).

### Example 10

### Preparation of intermediate chiral (2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid {4-[2-(tert-butyl-dimethyl-silanyloxy)-ethoxy]-2-methoxy-phenyl}-amide

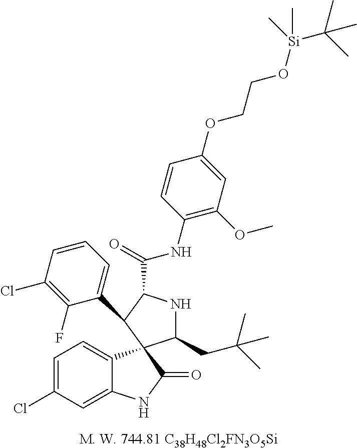

To a solution of chiral (2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid prepared in Example 8 (0.23 g, 0.4 mmol) in dichloromethane (7 mL) was added diisopropylethylamine (0.26 g, 1.9 mmol), diphenylphosphinic chloride (Aldrich) (0.19 g, 0.79 mmol) respectively. The mixture was stirred at room temperature for 8 min, then 4-[2-(tert-butyl-dimethyl-silanyloxy)-ethoxy]-2-methoxy-phenylamine (0.19 g, 0.6 mmol) was added. The reaction mixture was stirred at room temperature for 20 h. The mixture was concentrated. The residue was partitioned between ethyl acetate and water. The organic layer was separated, washed with water, brine, dried over Na₂SO₄, then concentrated. The residue was purified by chromatography (0-25% of EtOAc in CH₂Cl₂) to give chiral (2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid {4-[2-(tert-butyl-dimethyl-silanyloxy)-ethoxy]-2-methoxy-phenyl}-amide as a yellow solid (0.1 g, 34%).

### Example 11

### Preparation of chiral (3R,5′S,7′S,7a′R)-6-chloro-7′-(3-chloro-2-fluorophenyl)-2′-[4-(2-hydroxyethoxy)-2-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione

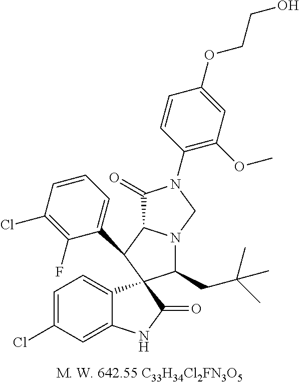

To a solution of chiral (2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid {4-[2-(tert-butyl-dimethyl-silanyloxy)-ethoxy]-2-methoxy-phenyl}-amide (0.1 g, 0.13 mmol) in DME (5 mL) was added an aqueous solution (Aldrich, 37%) of formaldehyde (0.54 g, 6.7 mmol), followed by the addition of an aqueous HCl solution (0.041 mL, 1 M, 0.04 mmol). The reaction mixture was heated at 60° C. for 6 h. The mixture was cooled to room temperature and concentrated. The residue was partitioned between ethyl acetate and water. The organic layer was separated, and aqueous layer was extracted with ethyl acetate. The combined organic extract was washed with water, brine, dried over Na₂SO₄, then concentrated. The residue was purified by R-HPLC (50-100% AcCN in water) to give chiral (3R,5′S,7′S,7a′R)-6-chloro-7′-(3-chloro-2-fluorophenyl)-2′-[4-(2-hydroxyethoxy)-2-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione as a white solid (23 mg, 27%).

LCMS (ES⁺) m/z Calcd for C₃₃H₃₄Cl₂FN₃O₅+H [(M+H)⁺]: 642, found: 642,

### Example 12

### Preparation of intermediate acetic acid 2-(4-amino-2-methoxy-phenoxy)-ethyl ester

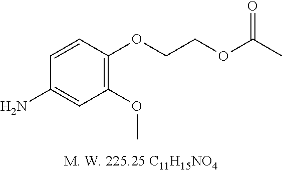

### Step A.

To a solution of 2-methoxy-4-nitrophenol (5 g, 30 mmol) in anhydrous DMF (50 mL) were added K₂CO₃ (6.6 g, 47 mmol) and (2-bromoethoxy)(tert-butyl)dimethylsilane (8.5 g, 36 mmol) sequentially. The reaction mixture was heated at 70° C. for 20 h. The mixture was cooled to room temperature, and diluted with water. The mixture was extracted with ethyl acetate three times. The combined organic extract was washed with water, brine, dried over MgSO₄, and concentrated to give tert-butyl-[2-(2-methoxy-4-nitro-phenoxy)-ethoxy]-dimethyl-silane as a brown oil (9 g, 93%).

### Step B.

To a solution of tert-butyl-[2-(2-methoxy-4-nitro-phenoxy)-ethoxy]-dimethyl-silane (9 g, 27.5 mmol) in THF (10 mL) was added an aqueous HCl solution (2N, 10 mL, 20 mmol). The reaction mixture was stirred at room temperature for 2 h. The mixture was concentrated. The residue was partitioned between ethyl acetate and saturated aqueous NaHCO₃ solution. The organic layer was separated, and aqueous layer was extracted with ethyl acetate. The combined organic extract was washed with water, brine, dried over MgSO₄, and concentrated to give 2-(2-methoxy-4-nitrophenoxy)ethanol as a off white solid (5.5 g, 94%).

### Step C.

To a solution of 2-(2-methoxy-4-nitrophenoxy)ethanol (5.5 g, 26 mmol) and pyridine (2.35 g, 30 mmol) in THF (140 mL) at 0° C. was acetyl chloride (2.33 g, 30 mmol). The reaction mixture was warmed to room temperature and stirred for 1 h. The mixture was concentrated. The residue was partitioned between ethyl acetate and water. The organic layer was separated, and aqueous layer was extracted with ethyl acetate. The combined organic extract was washed with water, saturated aqueous CuSO₄ solution, brine, dried over MgSO₄, and concentrated to give 2-(2-methoxy-4-nitrophenoxy)ethyl acetate as a yellow solid (6 g, 91%).

### Step D.

A suspension of 2-(2-methoxy-4-nitrophenoxy)ethyl acetate (1.5 g, 5.9 mmol) and Pd/C (Aldrich, 10%, 0.2 g) in ethyl acetate (19 mL) was vigorously shaken in a Parr under atmosphere of H₂ (50 psi) for 2 h. The mixture was filtered through a short pad of celite. The filtrate was concentrated to give acetic acid 2-(4-amino-2-methoxy-phenoxy)-ethyl ester as a light brown oil (1.32 g, 91%).

### Example 13

### Preparation of intermediate chiral acetic acid 2-(4-{[(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidin]e-5′-carbonyl]-amino}-2-methoxy-phenoxy)-ethyl ester

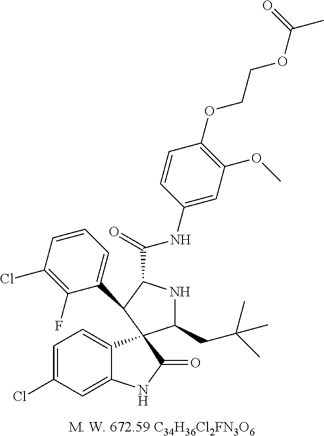

To a solution of chiral (2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidine]-5′-carboxylic acid trifluoroacetic acid prepared in Example 8 (0.4 g, 0.7 mmol) in dichloromethane (3 mL) was added diisopropylethylamine (0.45 g, 3.5 mmol), diphenylphosphinic chloride (Aldrich) (0.33 g, 1.4 mmol) respectively. The mixture was stirred at room temperature for 8 min, then acetic acid 2-(4-amino-2-methoxy-phenoxy)-ethyl ester (0.23 g, 1 mmol) was added. The reaction mixture was stirred at room temperature for 20 h. The mixture was concentrated. The residue was partitioned between ethyl acetate and water. The organic layer was separated, washed with water, brine, dried over Na₂SO₄, then concentrated. The residue was purified by chromatography (0-15% of EtOAc in CH₂Cl₂) to give chiral acetic acid 2-(4-{[(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidin]e-5′-carbonyl]-amino}-2-methoxy-phenoxy)-ethyl ester as a off white solid (0.28 g, 60%).

### Example 14

### Preparation of chiral (3R,5′S,7′S,7a′R)-6-chloro-7′-(3-chloro-2-fluorophenyl)-2′-[4-(2-hydroxyethoxy)-3-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione

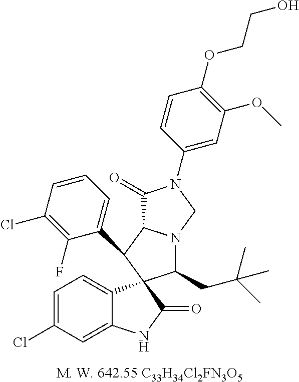

To a solution of chiral acetic acid 2-(4-{[(2′S,3′R,4′S,5′R)-6-chloro-4′-(3-chloro-2-fluoro-phenyl)-2′-(2,2-dimethyl-propyl)-2-oxo-1,2-dihydro-spiro[indole-3,3′-pyrrolidin]e-5′-carbonyl]-amino}-2-methoxy-phenoxy)-ethyl ester (0.24 g, 0.36 mmol) in DME (13 mL) was added an aqueous solution (Aldrich, 37%) of formaldehyde (1.45 g, 17.8 mmol), followed by the addition of an aqueous HCl solution (0.11 mL, 1 M, 0.11 mmol). The reaction mixture was heated at 60° C. for 6 h, then cooled to room temperature and stirred for 60 h. The mixture was concentrated, and the residue was partitioned between ethyl acetate and water. The organic layer was separated, and aqueous layer was extracted with ethyl acetate. The combined organic extract was washed with water, brine, dried over MgSO₄, and concentrated. The residue was purified by chromatography (0-100% EtOAc in dichlormethane) to give chiral (3R,5′S,7′S,7a′R)-6-chloro-7′-(3-chloro-2-fluorophenyl)-2′-[4-(2-hydroxyethoxy)-3-methoxyphenyl]-5′-(2,2-dimethyl-propyl)-2′,3′,7′,7a′-tetrahydrospiro[indoline-3,6′-pyrrolo[1,2-c]imidazole]-1′,2 (5′H)-dione as a white solid (65 mg, 28%).

LCMS (ES⁺) m/z Calcd for C₃₃H₃₄Cl₂FN₃O₅+H [(M+H)⁺]: 642, found: 642.

### Example 15

### Preparation of rac 5-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)picolinamide

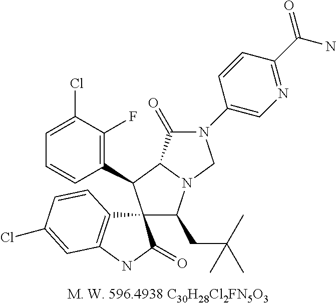

To a stirred solution of 5-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)picolinic acid (Example 40, 21 mg, 0.035 mmol) in methylene chloride (3 mL), HATU (Aldrich, 13.4 mg, 0.035 mmol) was added followed by DIPEA (Aldrich, 0.020 mL). The mixture was stirred at rt for 2 min followed by addition of ammonia in methanol (2N, 1.0 mL). The mixture was stirred at rt for 2 hours.

The solution was treated with 10 mL of water. The organic layer was separated and the aqueous layer was extracted with methylene chloride (2×5 mL). The organic parts were combined and dried with sodium sulfate and concentrated. The residue was purified on a reverse phase HPLC to give a white solid. 14 mg, yield 67%. LCMS (ES⁺) m/z Calcd for C₃₀H₂₈Cl₂FN₅O₃ [(M+H)⁺]: 596. found: 596.

### Example 16

### Preparation of Rac 4-((3R,5′S,7′S,7a′R)-6-chloro-7′-(3-chloro-2-fluorophenyl)-1-(hydroxymethyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid

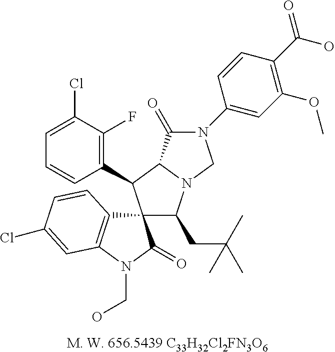

In a 20 mL sealed tube, 4-(2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)-2-methoxybenzoic acid (Prepared by a similar procedure as that for Example 10. 75 mg, 122 μmol) was combined with DME (5 mL) to give a colorless solution. Formaldehyde (198 mg, 182 μl, 2.44 mmol) in water was added and the reaction was stirred at 85° C. for 20 hr.

The reaction mixture was concentrated to remove some DME, added water and extracted with EtOAc (3×8 ml). The combined organic layer was dried over MgSO₄ and concentrated under vacuum. The crude material was purified by preparative HPLC (45-100 Acetonitrile/water, 0.1% TFA), concentrated and freeze dried to give a white solid. 26.5 mg, 33% yield. LCMS (ES⁺) m/z Calcd. for C₃₃H₃₂Cl₂FN₃O₆ [(M+H)⁺]: 656, found: 656.

### Example 17

### Preparation of Rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid

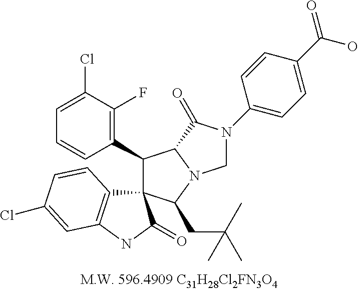

In a 20 mL scintillation vial, 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)benzoic acid (Prepared by a similar procedure as that for Example 10. 55 mg, 94.1 μmol) was combined with DME (4.00 ml) to give a light yellow solution. Formaldehyde in water (0.5 mL) was added, followed by a drop of 1M HCl (59.8 mg, 49.9 μl, 49.9 μmol). The reaction was stirred at 60° C. for 5 hr.

The reaction mixture was concentrated to remove some DME. Water was added (8 mL) and the mixture was extracted with EtOAc (3×10 mL). The organic layers were dried over Na₂SO₄ and concentrated under vacuum. The crude material was purified by preparative HPLC (50-100% ACN/water/0.1% TFA). The fractions were combined and freeze dried to give a white foam as desired product (20.6 mg, 36% yield). LCMS (ES⁺) m/z Calcd for C₃₁H₂₈Cl₂FN₃O₄ [(M+H)⁺]: 596, found: 596.

### Example 18

### Preparation of Rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl) 2-methoxybenzoic acid

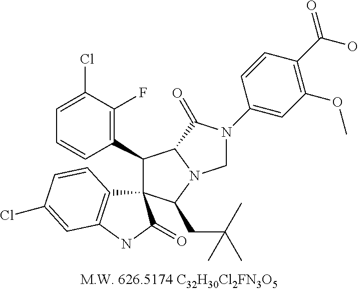

In a 20 mL scintillation vial, 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)-2-methoxybenzoic acid (Prepared by a similar procedure as that for Example 10. 100 mg, 163 μmol) was combined with DME (5.00 ml) to give a colorless solution. Formaldehyde in water (0.5 mL) was added and followed by a drop of 1M HCl (60.0 mg, 50 μl, 50.0 μmol). The reaction was stirred at 60° C. for 7 hr.

The reaction mixture was concentrated to remove some DME, water was added (8 mL) and the mixture was extracted with EtOAc (3×10 mL). The organic layers were dried over Na₂SO₄ and concentrated under vacuum. The crude material was purified by preparative HPLC (50-100% ACN/water/0.1% TFA). The fractions were combined and freeze dried to give a white foam as desired product (51.4 mg, 50% yield). LCMS (ES⁺) m/z Calcd for C₃₂H₃₀Cl₂FN₃O₅ [(M+H)⁺]: 626, found: 626.

### Example 19

### Preparation of chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl) 2-methoxybenzoic acid

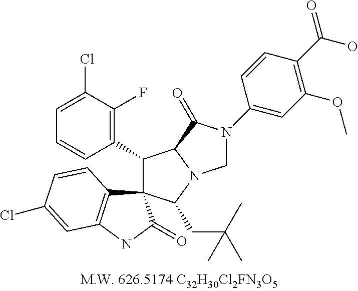

Rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)2-methoxybenzoic acid (40 mg) was separated by SFC with 35% MeOH+Triethylamime, 1000 BAR, OD01201012 (OD column):

Peak one was concentrated and freeze dried to give a powder as desired product. 16.4 mg, 82%.

LCMS (ES⁺) m/z Calcd for C₃₂H₃₀Cl₂FN₃O₅ [M+H)⁺]: 626; found: 626.

### Example 20

### Preparation of chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid

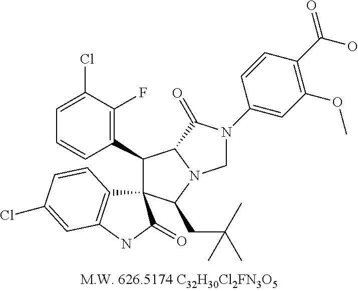

Rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)2-methoxybenzoic acid (40 mg) was separated by SFC with 35% MeOH+Triethylamime, 1000 BAR, OD01201012 (OD column):

Peak two was concentrated and freeze dried to give a powder as desired product. 18 mg, 89.4%.

LCMS (ES⁺) m/z Calcd for C₃₂H₃₀Cl₂FN₃O₅ [(M+H)⁺]: 626, found: 626.

### Example 21

### Preparation of chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid

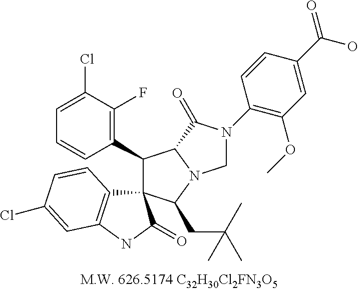

In a 20 mL scintillation vial, 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)-3-methoxybenzoic acid (Prepared by a similar procedure as that for Example 10. 150 mg, 244 μmol) was combined with DME (5.00 ml) to give a colorless solution. Formaldehyde in water (0.5 mL) was added, followed by 1M HCl. The reaction was stirred at 60° C. for 3 hr.

The reaction mixture was concentrated to remove some DME, water was added (8 mL) and the mixture was extracted with EtOAc (3×10 mL). The organic layers were dried over Na₂SO₄ and concentrated under vacuum to give the crude. This mixture was used directly for the next step.

In a 50 mL round-bottomed flask, the crude was combined with MeOH (10 ml) to give a colorless solution. 2 M NaOH (0.8 mL, 1.6 mmol, Eq: 13.1) was added and the reaction was stirred at rt for 3 h. To the reaction mixture was added 1N HCl to get a suspension. This was extracted with EtOAc (3×10 mL). The organic layers were combined, washed with H₂O (1×15 mL), sat NaCl (1×10 mL), dried over Na₂SO₄ and concentrated under vacuum.

The crude material was purified by preparative HPLC (40-90% Acetonitrile/Water/0.1% TFA). The fractions were combined, concentrated and freeze dried to give a white powder as desired product (93.5 mg, 62% yield). LCMS (ES⁺) m/z Calcd for C₃₂H₃₀Cl₂FN₃O₅ [(M+H)⁺]: 626, found: 626

### Example 22

### Preparation of chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzamide

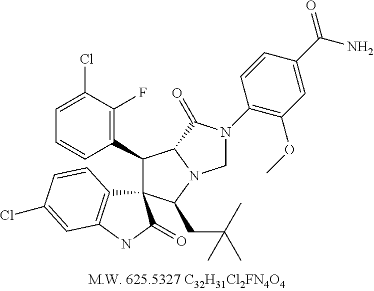

To a stirred solution of 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid (150 mg, 0.239 mmol) in methylene chloride (6 mL), HATU (Aldrich, 100 mg, 0.263 mmol) and DIPEA (0.1 ml, 0.57 mmol) were added and the mixture was stirred at rt for 5 min. The ammonia in methanol (2N, 1 mL) was added and the mixture was stirred overnight.

Treat with 1N HCl (5 mL) and stirred for 30 min. The organic layer was separated and dried with sodium sulfate. Concentrate to about 3 ml and separated on a combiflash (5% MeOH/methylene chloride) to give a white solid. 21 mg. LCMS (ES⁺) m/z Calcd for C₃₂H₃₁Cl₂FN₄O₄ [(M+H)⁺]: 625, found: 625.

### Example 23

### Preparation of chiral 4-((3R,5′S,7′S,7a′R)-6-chloro-7′-(3-chloro-4-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid

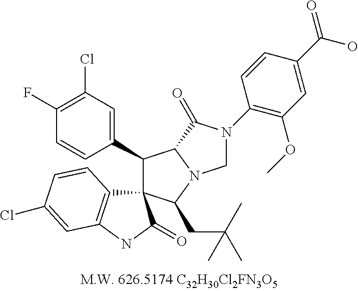

To a stirred solution of 4-((2′S,3R,4′S,5′R)-6-chloro-4′-(3-chloro-4-fluorophenyl)-2′-neopentyl-2-oxospiro[indoline-3,3′-pyrrolidine]-5′-ylcarboxamido)-3-methoxybenzoic acid (Prepared by a similar procedure as that for Example 10. 100 mg, 0.16 mmol) in DME (3 mL), formaldehyde (0.3 mL) was added and the mixture was stirred overnight. The solvent was removed and the residue was dissolved in 3 mL of methanol. 0.7 mL of 2N NaOH was added and the mixture was stirred overnight.

The solution was neutralized with 1 N HCl to PH=5 and extracted with THF/EtOAc (1:5). The extracts were dried with sodium sulfate and concentrated to give a white solid that was chromatographied to give an off white solid after removal of solvent. 57 mg. LCMS (ES⁺) m/z Calcd for C₃₂H₃₀Cl₂FN₃O₅ [(M+H)⁺]: 626, found: 626.

### Example 24

### Preparation of racl 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide

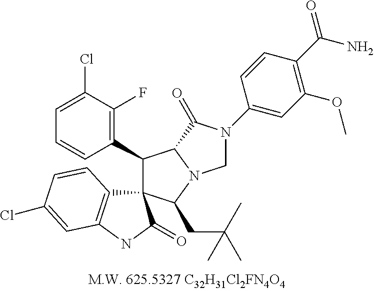

In a 10 mL round-bottomed flask, 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid (100 mg, 160 μmol) was combined with CH₂Cl₂ (4.00 mL) to give a suspension. DIPEA (41.3 mg, 55.8 μl, 319 μmol) and HATU (66.8 mg, 176 μmol) were added. The reaction was stirred for 2 minutes and 2M ammonia (399 μl, 798 μmol) in methanol was added. The reaction mixture was stirred for 2 h.

The crude material was purified by preparative HPLC (45-85% acetonitrile/water/0.1% TFA). The fractions (2nd peak) were combined to give a white solid as desired product (38.5 mg, 38% yield). LCMS (ES⁺) m/z for C₃₂H₃₁Cl₂FN₄O₄ [(M+H)⁺]: 625, found: 625.

### Example 25

### Preparation of chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide

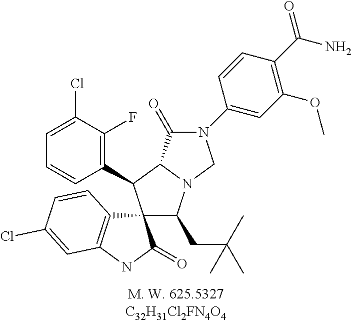

In a 10 mL round-bottomed flask, 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid (100 mg, 160 μmol) was combined with CH₂Cl₂ (4.00 ml) to give a suspension. DIPEA (41.3 mg, 55.8 μl, 319 μmol) and HATU (66.8 mg, 176 μmol) were added. The reaction was stirred for 2 minutes and 2M ammonia (399 μl, 798 μmol) in methanol was added. The reaction mixture was stirred for 2 h.

The crude material was purified by preparative HPLC (45-85% Acetonitrile/water/0.1% TFA). The fractions (2nd peak) were combined to give a white solid as desired product (38.5 mg, 38% yield). LCMS (ES⁺) m/z Calcd for C₃₂H₃₁Cl₂FN₄O₄ [(M+H)⁺]: 625, found: 625

The racemic mixture (76 mg) was separated by SFC (DAICEL OD2×25, T, 40° C.; MeOH 35%) chiral separation.

Peak 1 is the desired product, 33.4 mg, 89.4%. LCMS (ES⁺) m/z Calcd for C₃₂H₃₁Cl₂FN₄O₄ [(M+H)⁺]: 625, found: 625.

### Example 26

### Preparation of 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide

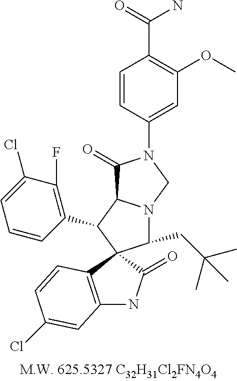

In a 10 mL round-bottomed flask, 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid (100 mg, 160 μmol) was combined with CH₂Cl₂ (4.00 ml) to give a suspension. DIPEA (41.3 mg, 55.8 μl, 319 μmol) and HATU (66.8 mg, 176 μmol) were added. The reaction was stirred for 2 minutes and 2M ammonia (399 μl, 798 μmol) in methanol was added. The reaction mixture was stirred for 2 h.

The crude material was purified by preparative HPLC (45-85% acetonitrile/water/0.1% TFA). The fractions (2nd peak) were combined to give a white solid as desired product (38.5 mg, 38% yield). LCMS (ES⁺) m/z Calcd for C₃₂H₃₁Cl₂FN₄O₄ [(M+H)⁺]: 625, found: 625.

The racemic mixture (76 mg) was separated by SFC (DAICEL OD2×25, T, 40° C.; MeOH 35%) chiral separation.

Peak 2 is the desired product, 31 mg, 82.9%. LCMS (ES⁺) Calcd for C₃₂H₃₁Cl₂FN₄O₄ [(M+H)⁺]: 625, found: 625.

### Example 27

### Preparation of rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzamide

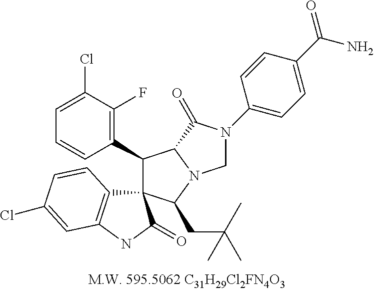

In a 15 mL round-bottomed flask, 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid (35 mg, 35.2 μmol) was combined with CH₂Cl₂ (3 mL) to give a suspension. DIPEA (9.1 mg, 12.3 μl, 70.4 μmol) and HATU (14.7 mg, 38.7 μmol, Eq: 1.1) were added. The reaction was stirred for 2 minutes and 2M ammonia (88.0 μl, 176 μmol) in methanol was added. The reaction mixture was stirred at rt overnight.

The reaction mixture was concentrated and the crude material was purified by preparative HPLC (45-100% ACN/water/0.1% TFA). The fractions were combined to give a white foam after freeze drying as desired product (13.2 mg, 62% yield).

LCMS (ES⁺) m/z Calcd for C₃₁H₂₉Cl₂FN₄O₃ [(M+H)^{+]:} 595, found: 595.

### Example 28

### Preparation of rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid

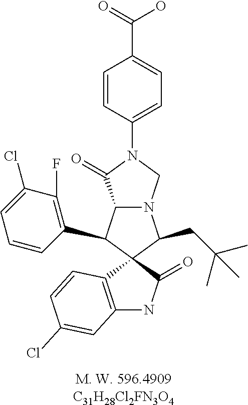

In a 20 mL scintillation vial, 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)benzoic acid (250 mg, 278 μmol) was combined with DME (11.3 ml) to give a light yellow solution. Formaldehyde in water (0.5 mL) was added, followed by a drop of 1M HCl (169 mg, 141 μl, 141 μmol). The reaction was stirred at 60° C. for 5 hr.

The reaction was diluted with EtOAC (15 mL). Water (10 mL) was added. The mixture was stirred and the layers were separated. The aqueous layer was extracted with EtOAc (2×10 mL). The organic layers were combined, washed with sat. NaCl (1×10 mL), dried over Na₂SO₄ and concentrated under vacuum. Obtained was a light yellow residue (102 mg).

7 mg of the above crude was purified by preparative HPLC (45-90% ACN/water/0.1% TFA). The fractions were combined and freeze dried to give a white foam as desired product (38.6 mg, 23%). LCMS (ES⁺) m/z Calcd for C₃₁H₂₈Cl₂FN₃O₄ [(M+H)⁺]: 596, found: 596.

### Example 29

### Preparation of chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid

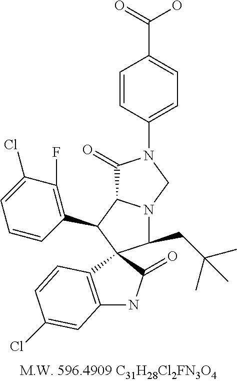

In a 20 mL scintillation vial, 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)benzoic acid (Prepared by a similar procedure as that for Example 10. 250 mg, 278 μmol) was combined with DME (11.3 ml) to give a light yellow solution. Formaldehyde in water (0.5 mL) was added, followed by a drop of 1M HCl (169 mg, 141 μl, 141 μmol). The reaction was stirred at 60° C. for 5 hr. completion of reaction.

The reaction was diluted with EtOAC (15 mL). Water (10 mL) was added. The mixture was stirred and the layers were separated. The aqueous layer was extracted with EtOAc (2×10 mL). The organic layers were combined, washed with sat. NaCl (1×10 mL), dried over Na₂SO₄ and concentrated under vacuum.

Obtained was a light yellow residue (102 mg).

67 mg of the above crude was purified by preparative HPLC (45-90% ACN/water/0.1% TFA). The fractions were combined and freeze dried to give a white foam as desired product (38.6 mg, 23% yield). LCMS (ES⁺) Calcd for C₃₁H₂₈Cl₂FN₃O₄ [(M+H)⁺]: 596, found: 596.

Separation by SFC (25% MeOH) on an OJ column give the desired product. 13.6 mg, 76%. LCMS (ES⁺) Calcd for C₃₁H₂₈Cl₂FN₃O₄ [(M+H)⁺]: 596, found: 596.

### Example 30

### Preparation of chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid

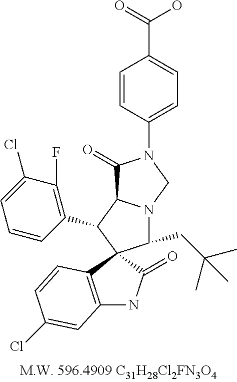

In a 20 mL scintillation vial, 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)benzoic acid (Prepared by a similar procedure as that for Example 10. 250 mg, 278 μmol) was combined with DME (11.3 ml) to give a light yellow solution. Formaldehyde in water (0.5 mL) was added, followed by a drop of 1M HCl (169 mg, 141 μl, 141 μmol). The reaction was stirred at 60° C. for 5 hr.

The reaction was diluted with EtOAC (15 mL). Water (10 mL) was added. The mixture was stirred and the layers were separated. The aqueous layer was extracted with EtOAc (2×10 mL). The organic layers were combined, washed with sat. NaCl (1×10 mL), dried over Na₂SO₄ and concentrated under vacuum. Obtained was a light yellow residue (102 mg).

67 mg of the above crude was purified by preparative HPLC (45-90% ACN/water/0.1% TFA). The fractions were combined and freeze dried to give a white foam as desired product (38.6 mg, 23% yield). LCMS (ES⁺) m/z Calcd for C₃₁H₂₈Cl₂FN₃O₄ [(M+H)⁺]: 596, found: 596.

Separation by SFC (25% MeOH) on an OJ column give the desired product. 13.7 mg, 76%. LCMS (ES⁺) Calcd for C₃₁H₂₈Cl₂FN₃O₄ [(M+H)⁺]: 596, found: 596.

### Example 31

### Preparation of 4-((3R,5′S,7′S,7a′R)-6-chloro-7′-(3-chloro-2-fluorophenyl)-1-(hydroxymethyl)-5-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide

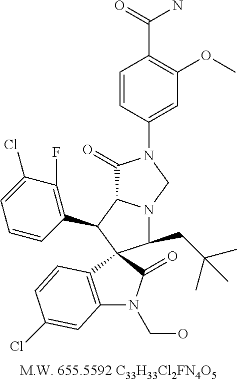

In a 10 mL round-bottomed flask, 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid (100 mg, 160 μmol) was combined with CH₂Cl₂ (4.00 ml) to give a suspension. DIPEA (41.3 mg, 55.8 μl, 319 μmol) and HATU (66.8 mg, 176 μmol) were added. The reaction was stirred for 2 minutes and 2M ammonia (399 μl, 798 μmol) in methanol was added. The reaction mixture was stirred for 2 hrs.

The crude material was purified by preparative HPLC (45-85% Acetonitrile/water/0.1% TFA. The fractions were combined and evaporated to give a white solid as desired product. 38.5 mg, 38% yield. LCMS (ES⁺) m/z Calcd for C₃₃H₃₃Cl₂FN₄O₅ [(M+H)⁺]: 655, found: 655.

### Example 32

### Preparation of rac methyl 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoate

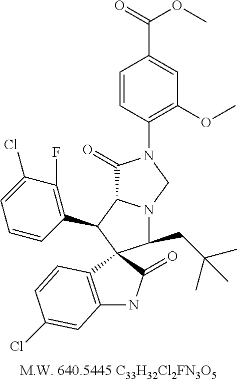

In a 20 mL scintillation vial, methyl 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)-3-methoxybenzoate (Prepared by a similar procedure as that for Example 10. 100 mg, 159 μmol) was combined with DME (3.33 mL) to give a colorless solution. Formaldehyde in water (0.5 mL) was added and followed by 1M HCl (175 μL, 175 μmol). The reaction was stirred at 50° C. for 3 hr.

To the reaction mixture was added water (8 mL), and extracted with EtOAc (3×10 mL). The organic layers were dried over Na₂SO₄ and concentrated in vacuum. The crude material was purified by flash chromatography (silica gel, 24 g, 5% to 20% EtOAc in DCM). The fractions were combined and concentrated and freeze dried to give a white powder (48 mg, 47% yield) as desired product. LCMS (ES⁺) m/z Calcd for C₃₃H₃₂Cl₂FN₃O₅ [(M+H)⁺]: 640, found: 640.

### Example 33

### Preparation of rac methyl 4-((3R,5′S,7′S,7a′R)-6-chloro-7′-(2,5-difluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-fluorobenzoate

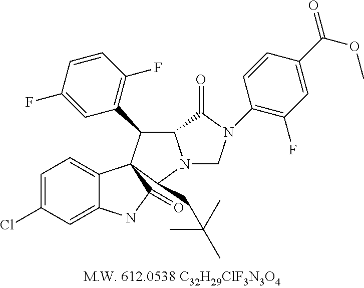

By a similar procedure to the preparation of rac methyl 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoate

(example 32), rac methyl 4-((3R,5′S,7′S,7a′R)-6-chloro-7′-(2,5-difluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-fluorobenzoate was prepared. LCMS (ES⁺) m/z Calcd for C₃₂H₂₉ClF₃N₃O₄ [(M+H)⁺]: 612, found: 612.

### Example 34

### Preparation of rac 2-chloro-4-((3R,5′S,7′R,7a′R)-7′-(3-chlorophenyl)-6-fluoro-5′-neopentyl-1′,2-dioxo-7′,7a′-dihydrospiro[indoline-3,6′-pyrrolo[1,2-c]imidazole]-2′(1′H,3′H,5′H)-yl)benzamide

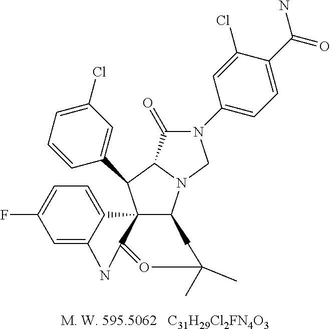

By a similar procedure to the preparation of rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzamide (example 27), rac methyl 4-(3R,5′S,7′S,7a′R)-6-chloro-7′-(2,5-difluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-fluorobenzoate was prepared. LCMS (ES⁺) m/z Calcd for C₃₁H₂₉Cl₂FN₄O₃ [(M+H)⁺]: 595, found: 595.

### Example 35

### Preparation of rac {[(2′S,3′R,4′S,5′R)-6-Chloro-4′-(3-chloro-2-fluoro-phenyl)-2′-(2,2-dimethyl-propyl)-2-oxo-1,2-dihydro-spiro[indole-3,3′-pyrrolidin]e-5′-carbonyl]-amino-acetic acid tert-butylester

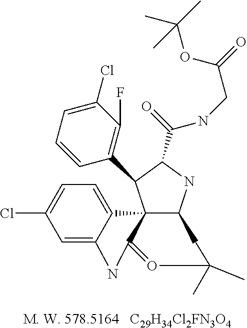

To a stirred solution of (2′S,3R,4′S,5′R)-6-chloro-4′-(3-chloro-2-fluorophenyl)-2′-neopentyl-2-oxospiro[indoline-3,3′-pyrrolidine]-5′-carboxylic acid (Example 8, 240 mg, 0.516 mmol) in methylene chloride (7 mL), HATU (196 mg, 0.516 mmolo) and DIPEA (0.516 mmol) were added and the mixture was stirred at rt for 3 min. followed by tert-butyl 2-aminoacetate (68 mg, 0.516 mmol). The mixture was stirred at rt for 30 min. Then chromatographied on a combiflash machine (5-30 EtOAc/methylene chloride) to a give a white solid. 178 mg. 60%.

LCMS (ES⁺) m/z Calcd for C₂₉H₃₄Cl₂FN₃O₄ [(M+H)⁺]: 578, found: 578.

### Example 36

### Preparation of rac {[(2′S,3′R,4′S,5′R)-6-Chloro-4′-(3-chloro-2-fluoro-phenyl)-2′-(2,2-dimethyl-propyl)-2-oxo-1,2-dihydro-spiro[indole-3,3′-pyrrolidin]e-5′-carbonyl]-amino}-acetic acid

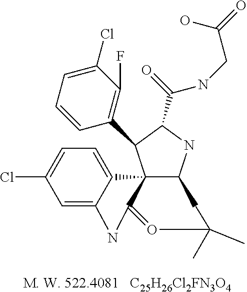

To a stirred solution of tert-butyl 2-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)acetate (168 mg, 0.29 mmol) in methylene chloride (4 mL), TFA (2 mL) was added and the mixture was stirred at rt overnight. The solvent was removed and the residue was triturated with ether to give a white solid. 180 mg, 97%. LCMS (ES⁺) m/z Calcd for C₂₅H₂₆Cl₂FN₃O₄ [(M+H)⁺]: 522, found: 522

### Example 37

### Preparation of rac of 2-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)acetic acid

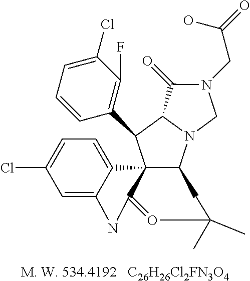

To a stirred solution of 2-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)acetic acid trifluoroacetic acid salt (160 mg, 0.25 mmol) in 3 mL of acetic acid, formaldehyde (0.30 mmol, 37% in water, 0.024 mL) was added and the mixture was stirred at 70° C. for 2 hrs.

The solvent was removed under reduced pressure and the residue was triturated with ether to give a white solid. 144 mg. The crude was re-dissolved in 3 mL of methanol and 0.5 mL of 2 N NaOH was added. The mixture was stirred at rt for 2 hrs and the solution was neutralized with 1 N HCl to PH=5. The solid was filtered and dried to give a white solid. 97 mg, 59%. LCMS (ES⁺) m/z Calcd for C₂₆H₂₆Cl₂FN₃O₄ [(M+H)⁺]: 534, found: 534.

### Example 38

### Preparation of rac 5-{[(2′S,3′R,4′S,5′R)-6-Chloro-4′-(3-chloro-2-fluoro-phenyl)-2′-(2,2-dimethylpropyl)-2-oxo-1,2-dihydro-spiro[indole-3,3′-pyrrolidin]e-5′-carbonyl]-amino}-pyridine-2-carboxylic acid methyl ester

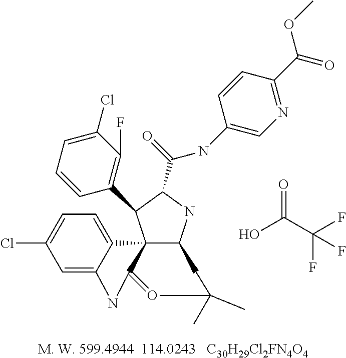

In a 20 mL round-bottomed flask, (2′S,3R,4′S,5′R)-6-chloro-4′-(3-chloro-2-fluorophenyl)-2′-neopentyl-2-oxospiro[indoline-3,3′-pyrrolidine]-5′-carboxylic acid 2,2,2-trifluoroacetic acid salt (500 mg, 863 μmol) was combined with CH₂Cl₂ (12.5 mL) to give a suspension, DIPEA (Aldrich, 558 mg, 754 μl, 4.32 mmol) and diphenylphosphinic chloride (Aldrich, 715 mg, 575 μl, 3.02 mmol) were added under N₂. The reaction was stirred at rt for 5 minutes, methyl 5-aminopicolinate (263 mg, 1.73 mmol) was added and the reaction mixture was stirred at RT overnight.

The reaction mixture was diluted with methylene chloride (25 washed with dilute HCl (0.1 M, 15 mL), water (15 mL) and brine (10 mL), dried over MgSO₄ and concentrated in vacuo to give a yellow foam.

The foam was dissolved in CH₂Cl₂. A solid precipitated out which was filtered out. The mother liquor was purified by flash chromatography (silica gel, 50 g, 5% to 20% EtOAc in methylene chloride, then 2-5% MeOH/methylene chloride). The fractions were combined and concentrated to give a white solid which contaminated with a little methyl 5-aminopicolinate (0.52 g). 40 mg of the crude was further purified on a reverse phase HPLC to give an analytically pure product. LCMS (ES⁺) m/z Calcd for C₃₀H₂₉Cl₂FN₄O₄ [(M+H)⁺]: 599, found: 599.

### Example 39

### Preparation of rac 5-{[(2′S,3′R,4′S,5′R)-6-Chloro-4′-(3-chloro-2-fluoro-phenyl)-2′-(2,2-dimethylpropyl)-2-oxo-1,2-dihydro-spiro[indole-3,3′-pyrrolidin]e-5′-carbonyl]-amino}-pyridine-2-carboxylic acid

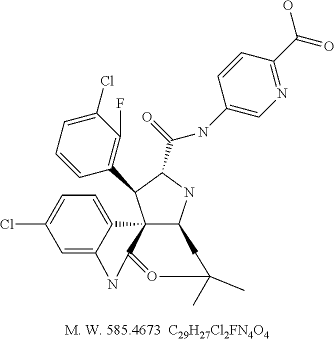

In a 20 mL round-bottomed flask, methyl 5-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)picolinate (400 mg, 667 μmol) was combined with THF (5 ml) to give a colorless solution. 2M NaOH (2 mL, 4.00 mmol) was added and the reaction was stirred at 43° C. for 2 hr.

The reaction mixture was concentrated to remove most of the solvent, diluted with water (10 mL). To the mixture, 1N HCl was added to pH=3 to obtain a suspension. This was filtered to collect the solid. Obtained was a light yellow solid as crude product, 150 mg, that was used directly for the next step.

### Example 40

### Preparation of rac 5-{[(2′S,3′R,4′S,5′R)-6-Chloro-4′-(3-chloro-2-fluoro-phenyl)-2′-(2,2-dimethylpropyl)-2-oxo-1,2-dihydro-spiro[indole-3,3′-pyrrolidin]e-5′-carbonyl]-amino}-pyridine-2-carboxylic acid

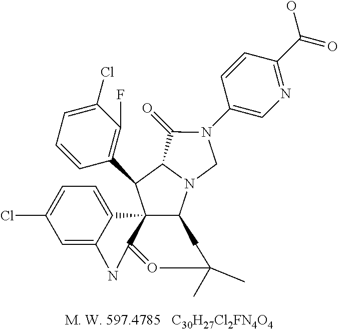

In a 20 mL scintillation vial, 5-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)picolinic acid (85 mg, 145 μmol) was combined with THF (4 mL) to give a colorless solution. Formaldehyde (471 mg, 432 μl, 5.81 mmol) was added. The reaction mixture was heated to 50° C. and stirred for 3 hrs. The reaction mixture was poured into 10 mL H₂O and extracted with EtOAc (3×10 mL). The organic layers were combined, washed with sat. NaCl (2×10 mL), dried over Na₂SO₄ and concentrated in vacuo to give a white solid (80 mg). The solid was dissolved in methanol (5 mL), 2N NaOH (0.40 mL) was added and the mixture was stirred at rt for 45 min.

The mixture was adjusted to PH=4 by adding 1N HCl. The solvent was removed under reduced pressure to give a white solid, which was purified on reverse phase HPLC to give a white solid. 48 mg, yield, 60%. LCMS (ES⁺) m/z Calcd for C₃₀H₂₇Cl₂FN₄O₄ [(M+H)⁺]: 598, found: 598.

### Example 41

### Preparation of 4-((3S,3′S,5′S,7′R,7a′R)-6-chloro-7′-(3-chloro-4-fluorophenyl)-3′-cyclopropyl-5′-neopentyl-1′,2-dioxo-7′,7a′-dihydrospiro[indoline-3,6′-pyrrolo[1,2-c]imidazole]-2′(1′H,3′H,5′H)-yl)benzamide

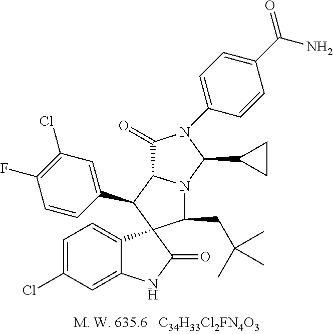

To a 10 mL microwave vial was added (2′S,3R,4′R,5′R)—N-(4-carbamoylphenyl)-6-chloro-4′-(3-chloro-4-fluorophenyl)-2′-neopentyl-2-oxospiro[indoline-3,3′-pyrrolidine]-5′-carboxamide (Prepared by a similar procedure as that for example 10. 40 mg, 0.068 mmol), cyclopropanecarbaldehyde (4.8 mg, 68.6 μmol) and in AcOH (833 μl) and CH₂Cl₂ (1 mL) to give a colorless solution. The vial was capped and heated at 90° C. overnight. The reaction mixture was quenched with 2.0N NaOH and extracted with CH₂Cl₂ (3×20 mL). The organic layers were dried over Na₂SO₄ and concentrated in vacuo. The crude material was purified by flash chromatography (silica gel, 4 g, 1% to 20% EtOAc in hexanes) to give 4-((3S,3′S,5′S,7′R,7a′R)-6-chloro-7′-(3-chloro-4-fluorophenyl)-3′-cyclopropyl-5′-neopentyl-1′,2-dioxo-7′,7a′-dihydrospiro[indoline-3,6′-pyrrolo[1,2-c]imidazole]-2′(1′H,3′H,5′H)-yl)benzamide (4.6 mg, 10.6%) as a white amorphous: HRMS (ES⁺) m/z Calcd C₃₄H₃₃Cl₂FN₄O₃+H [(M+H): 635.1987, found: 635.1987.

### Example 42

### Preparation of rac 4-((3S,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide and 4-((3R,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide

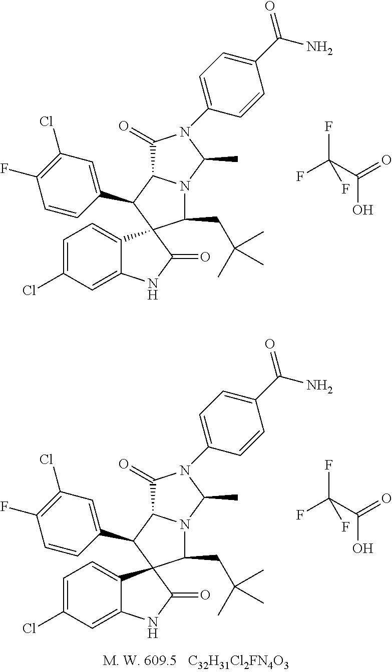

To a 10 mL microwave vial was added (2′S,3R,4′R,5′R)—N-(4-carbamoylphenyl)-6-chloro-4′-(3-chloro-4-fluorophenyl)-2′-neopentyl-2-oxospiro[indoline-3,3′-pyrrolidine]-5′-carboxamide (Prepared by a similar procedure as that for example 10. 50 mg, 85.7μmol), acetaldehyde (75.5 mg, 1.71 mmol) and in AcOH (833 μl) and DCM (1 ml) to give a colorless solution. The vial was capped and heated in the microwave at 120° C. for 15 min. The reaction mixture was quenched with 2.0N NaOH and extracted with CH₂Cl₂ (3×20 mL). The organic layers were dried over Na₂SO₄ and concentrated in vacuo. The crude material was purified by preparative RP-HPLC to give 4-((3S,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide (13.8 mg, 22.3%) as a TFA salt, HRMS (ES⁺) m/z Calcd C₃₄H₃₃Cl₂FN₄O₃+H [(M+H): 609.1830, found: 609.1832.

¹H NMR (400 MHz, CHLOROFORM-d) d ppm 7.90 (d, J=8.8 Hz, 2H) 7.74 (d, J=8.8 Hz, 2H) 7.48-7.60 (m, 1H) 7.18-7.26 (m, 2H) 7.09 (dd, J=8.0, 1.8 Hz, 1H) 6.92-6.96 (m, 2H) 6.75 (d, J=1.8 Hz, 1H) 6.27 (br. s., 2H) 5.26 (q, J=5.9 Hz, 1H) 4.79 (d, J=8.3 Hz, 1H) 3.82 (d, J=8.3 Hz, 1H) 3.59 (t, J=4.2 Hz, 1H) 1.70 (d, J=4.2 Hz, 2H) 1.55 (d, J=5.9 Hz, 3H) 0.66 (s, 9H), and 4-((3R,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide (13.4 mg, 21.6%) as a TFA salt, HRMS (ES⁺) m/z Calcd C₃₄H₃₃Cl₂FN₄O₃+H [(M+H): 609.1830, found: 609.1832. ¹H NMR (400 MHz, CHLOROFORM-d) d ppm 7.89 (d, J=8.8 Hz, 2H) 7.85-7.97 (m, 1H) 7.73 (d, J=8.8 Hz, 2H) 7.57 (d, J=8.0 Hz, 1H) 7.08 (td, J=7.5, 2.9 Hz, 2H) 6.83-6.92 (m, 2H) 6.75 (d, J=1.9 Hz, 1H) 5.80-6.29 (m, 2H) 5.23 (q, J=6.0 Hz, 1H) 4.44 (d, J=8.4 Hz, 1H) 4.14 (d, J=8.4 Hz, 1H) 3.68 (dd, J=5.4, 3.1 Hz, 1H) 1.53 (d, J=6.0 Hz, 3H) 1.22-1.37 (m, 1H) 0.82 (s, 9H) 0.57-0.69 (m, 1H)

### Example 43

### Preparation of rac 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid hydrochloride

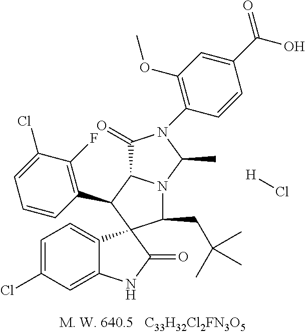

To a 10 mL microwave vial was added methyl 4-((2′S,3R,4′S,5′R)-6-chloro-4′-(3-chloro-2-fluorophenyl)-2′-neopentyl-2-oxospiro[indoline-3,3′-pyrrolidine]-5′-ylcarboxamido)-3-methoxybenzoate (Prepared by a similar procedure as that for example 10. 60 mg, 95.5 μmol), acetaldehyde (42.1 mg, 955 μmol) and in AcOH (1.00 ml) and CH₂Cl₂ (1 mL) to give a colorless solution. The vial was capped and heated in the microwave at 120° C. for 15 min. The reaction mixture was quenched with 2.0N NaOH and extracted with CH₂Cl₂ (3×20 mL). The organic layers were dried over Na₂SO₄ and concentrated in vacuo. The crude material was purified by flash chromatography (silica gel, 4 g, 1% to 20% EtOAc in hexanes) to give 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid methyl ester (22.6 mg, 36.2%).

In a 25 mL round-bottomed flask, methyl 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid methyl ester (20.3 mg, 0.031 mmol) was combined with THF (1.8 ml) and MeOH (0.60 ml) to give a light yellow solution. To this solution 2.0N KOH (0.60 ml) was added. The reaction mixture was stirred at rt 4 hrs. The reaction mixture was quenched with 0.5 mL 1 M HCl and extracted with EtOAc (3×25 mL). The organic layers were combined, washed with H₂O (1×10 mL), sat NaCl (1×10 mL), and dried over Na₂SO₄ and concentrated in vacuo without further purification to give 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid hydrochloride as a white powder (19.2 mg, 91.4%). HRMS (ES⁺) m/z Calcd C₃₃H₃₂Cl₂FN₃O₅+H [(M+H): 640.1776, found: 640.1773

### Example 44

### In Vitro Activity Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53. Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

Test is performed in black flat-bottom 384-well plates (Costar) in a total volume of 40 uL containing: 90 nM biotinylate peptide, 160 ng/ml GST-MDM2, 20 nM streptavidin-APC (PerkinElmerWallac), 2 nM Eu-labeled anti-GST-antibody (PerkinElmerWallac), 0.2% bovine serum albumin (BSA), 1 mM dithiothreitol (DTT) and 20 mM Tris-borate saline (TBS) buffer as follows: Add 10 uL of GST-MDM2 (640 ng/ml working solution) in reaction buffer to each well. Add 10 uL diluted compounds (1:5 dilution in reaction buffer) to each well, mix by shaking Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.2% BSA, shake at room temperature for 30 minutes and read using a TRF-capable plate reader at

665 and 615 nm (Victor 5, Perkin ElmerWallac). If not specified, the reagents were purchased from Sigma Chemical Co.

Activity data for some of the Example compounds expressed as IC₅₀: bsa: 0.02% are as follows:

| Example Number | IC₅₀: bsa: 0.02% (IC₅₀, μM) |
|---|---|
| Example 16 | 0.025 |
| Example 18 | 0.007 |
| Example 21 | 0.006 |
| Example 22 | 0.005 |
| Example 25 | 0.004 |
| Example 30 | 0.128 |
| Example 31 | 0.012 |
| Example 37 | 0.013 |

What is claimed:

1. A compound of the formula 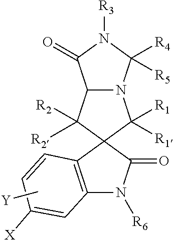
wherein
X is selected from the group consisting of H, F, Cl, Br, I, cyano, nitro, lower alkyl, lower alkynyl and lower alkoxy.
Y is H or F;
R₁ and R_{1′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ and R_{2′} are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and a pharmaceutically acceptable salt thereof.

2. The compound of claim 1 wherein R_{2′} is H, R₂ is a substituted phenyl as shown in formula II 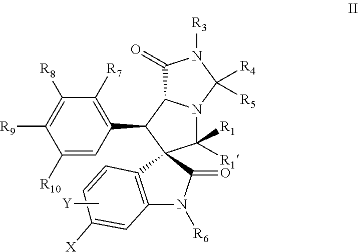
X is selected from the group consisting of H, F, Cl, Br, I, cyano, nitro, lower alkyl, lower alkynyl and lower alkoxy;
Y is H or F;
R₈ is selected from the group consisting of F, Cl and Br;
R₇, R₉ and R₁₀ are H or F with the proviso that at least two of R₇, R₉ and R₁₀ are hydrogen;
R₁ and R_{1′} are independently selected from the group consisting of hydrogen, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group; R₆ is selected from the group consisting of hydrogen, lower alkyl and substituted lower alkyl
and the enantiomers thereof or a pharmaceutically acceptable salt or ester thereof.

3. The compound of claim 2 wherein R_{1′} is hydrogen, R₁ is a substituted lower alkyl shown as in formula III: 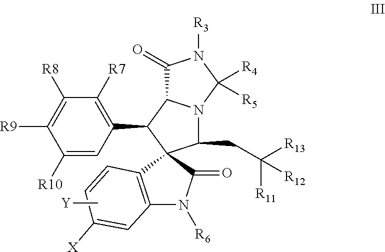
X is selected from the group consisting of H, F, Cl, Br, I, cyano, nitro, lower alkyl, lower alkynyl and lower alkoxy;
Y is H or F;
R₈ is selected from the group consisting of F, Cl and Br;
R₇, R₉, R₁₀ are selected from H or F with the proviso that at least two of R₇, R₉ and R₁₀ are hydrogen;
R₁₁, R₁₂ are both methyl, or linked to form a cyclopropyl, cyclobutyl, cyclopentyl or cyclohexyl group;
R₁₃ is (CH₂)ₘ—R₁₄;
m is selected from 0, 1 or 2;
R₁₄ is selected from the group consisting of hydrogen, hydroxyl, lower alkyl, lower alkoxy, lower cycloalkenyl, substituted cycloalkenyl, lower cycloalkyl, substituted cycloalkyl, aryl, substituted aryl, hetereoaryl, substituted heteroaryl, hetereocycle and substituted heterocycle;
R₃ is aryl, substituted aryl, heteroaryl or substituted heteroaryl;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and enantiomers thereof or a pharmaceutically acceptable salt thereof.

4. The compound of claim 3 wherein R₁₁, R₁₂, R₁₃ are methyl as shown in formula IV, 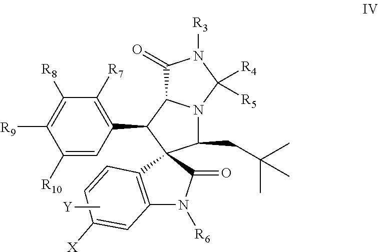
X is selected from the group consisting of H, F, Cl, Br, I, cyano, nitro, lower alkyl, lower alkynyl and lower alkoxy;
Y is H or F;
R₈ is selected from the group consisting of F, Cl and Br;
R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and the enantiomers thereof or a pharmaceutically acceptable salt or ester thereof.

5. The compound of claim 4 wherein R₆ is H as shown in formula V. 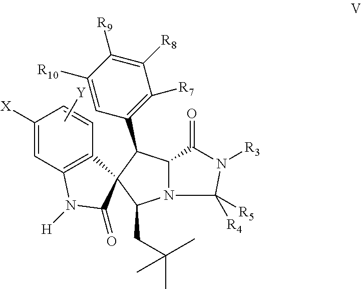
X is selected from the group consisting of F, Cl, Br
Y is H or F;
R₈ is selected from the group consisting of F, Cl and Br, R₇, R₉, R₁₀ is selected from H or F with the proviso that at least two of R₇, R₉, R₁₀ are hydrogen;
R₃ is selected from the group consisting aryl, substituted aryl, heteroaryl or substituted heteroaryl wherein the substituents are selected from H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thioxo group;
R₆ is selected from the group consisting of hydrogen, lower alkyl and substituted lower alkyl and the enantiomers thereof and a pharmaceutically acceptable salt thereof.

6. A compound of claim 1 selected from the group consisting of
rac 5-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)picolinamide,
rac-4-((3R,5′S,7′S,7a′R)-6-chloro-7′-(3-chloro-2-fluorophenyl)-5′-(2,2-dimethyl-propyl)-1′,2-dioxo-3′-thioxo-7′,7a′-dihydrospiro[indoline-3,6′-pyrrolo[1,2-c]imidazole]-2′ (1′H,3′H,5′H)-yl)benzonitrile,
rac-4-((3R,5′S,7′S,7a′R)-6-chloro-7′-(3-chloro-2-fluorophenyl)-5′-(2,2-dimethyl-propyl)-1′,2,3′-trioxo-7′,7a′-dihydrospiro[indoline-3,6′-pyrrolo[1,2-c]imidazole]-2′(1′H,3′H,5′H)-yl)benzonitrile,
chiral (3R,5′S,7′S,7a′R)-6-chloro-7′-(3-chloro-2-fluorophenyl)-2′-[4-(2-hydroxyethoxy)-2-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione,
chiral (3R,5′S,7′S,7a′R)-6-chloro-7′-(3-chloro-2-fluorophenyl)-2′-[4-(2-hydroxyethoxy)-3-methoxyphenyl]-5′-(2,2-dimethyl-propyl)-2′,3′,7′,7a′-tetrahydrospiro[indoline-3,6′-pyrrolo[1,2-c]imidazole]-1′,2(5′H)-dione,
rac4-((3R,5′S,7′S,7a′R)-6-chloro-7′-(3-chloro-2-fluorophenyl)-1-(hydroxymethyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl) 2-methoxybenzoic acid,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl) 2-methoxybenzoic acid,
chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzoic acid,
chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′ (1′H,3′H,5′H)-yl)-3-methoxybenzoic acid and
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′ (1′H,3′H,5′H)-yl)-3-methoxybenzamide.

7. A compound of claim 1 selected from the group consisting of
rac 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid hydrochloride,
chiral 4-((3R,5′S,7′S,7a′R)-6-chloro-7′-(3-chloro-4-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid,
racl 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzamide,
rac 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
chiral 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid,
chiral 4-((3S,5′R,7′R,7a′S)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)benzoic acid and
4-((3R,5′S,7′S,7a′R)-6-chloro-7′-(3-chloro-2-fluorophenyl)-1-(hydroxymethyl)-5′-neopentyl-1′,2-dioxo-7′,7a′-dihydrospiro[indoline-3,6′-pyrrolo[1,2-c]imidazole]-2′(1′H,3′H,5′H)-yl)-2-methoxybenzamide.

8. A compound of claim 1 selected from the group consisting of
rac methyl 4-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoate,
rac methyl 4-((3R,5′S,7′S,7a′R)-6-chloro-7′-(2,5-difluorophenyl)-5′-neopentyl-1′,2-dioxo-7′,7a′-dihydrospiro[indoline-3,6′-pyrrolo[1,2-c]imidazole]-2′ (1′H,3′H,5′H)-yl)-3-fluorobenzoate,
rac 2-chloro-4-((3R,5′S,7′R,7a′R)-7′-(3-chlorophenyl)-6-fluoro-5′-neopentyl-1′,2-dioxo-7′,7a′-dihydrospiro[indoline-3,6′-pyrrolo[1,2-c]imidazole]-2′(1′H,3′H,5′H)-yl)benzamide,
rac of 2-((3R,5′S,7′S,7a′R)-6-chloro-7′-(3-chloro-2-fluorophenyl)-5′-neopentyl-1′,2-dioxo-7′,7a′-dihydrospiro[indoline-3,6′-pyrrolo[1,2-c]imidazole]-2′(1′H,3′H,5′H)-yl)acetic acid,
rac 5-{[(2′S,3′R,4′S,5′R)-6-Chloro-4′-(3-chloro-2-fluoro-phenyl)-2′-(2,2-dimethylpropyl)-2-oxo-1,2-dihydro-spiro[indole-3,3′-pyrrolidin]e-5′-carbonyl]-amino}-pyridine-2-carboxylic acid,
4-((3S,3′S,5′S,7′R,7a′R)-6-chloro-7′-(3-chloro-4-fluorophenyl)-3′-cyclopropyl-5′-neopentyl-1′,2-dioxo-7′,7a′-dihydrospiro[indoline-3,6′-pyrrolo[1,2-c]imidazole]-2′(1′H,3′H,5′H)-yl)benzamide,
rac 4-((3S,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide,
4-((3R,3′S,5′S,7′R,7a′R)-6-chloro-7′-(3-chloro-4-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)benzamide and
rac 4-((3′S,5′S,7′S,7a′R)-6-chloro-7′-(3-chloro-2-fluorophenyl)-3′-methyl-5′-neopentyl-1′,2-dioxo-7′,7a′-dihydrospiro[indoline-3,6′-pyrrolo[1,2-c]imidazole]-2′(1′H,3′H,5′H)-yl)-3-methoxybenzoic acid hydrochloride.

9. A pharmaceutical composition comprising a compound of claim 1, or a pharmaceutically acceptable salt or ester thereof, as an active ingredient together with a pharmaceutically acceptable carrier or excipient.
